(19) United States
(12) Patent Application Publication
(10) Pub. No.: US 2020/0107812 A1
(43) Pub. Date: Apr. 9, 2020

(54) ULTRASONIC-BASED PULSE-TAKING DEVICE AND PULSE-TAKING METHOD

(71) Applicant: Acer Incorporated, New Taipei City (TW)

(72) Inventors: Jyun-Guo Wang, New Taipei City (TW); Yin-Hsong Hsu, New Taipei City (TW)

(73) Assignee: Acer Incorporated, New Taipei City (TW)

(21) Appl. No.: 16/209,989

(22) Filed: Dec. 5, 2018

(30) Foreign Application Priority Data
Oct. 4, 2018 (TW) 107135144

(51) Int. Cl. A61B 8/08; A61B 8/06

(52) U.S. Cl. CPC A61B 8/0891; G06T 2207/10132; A61B 8/488; A61B 8/06

(57) ABSTRACT

An ultrasonic-based pulse-taking device and a pulse-taking method thereof which are adapted to detect a pulse condition of a person are provided. The pulse-taking method includes: sensing a blood vessel of the person to generate a color Doppler image and a blood flow waveform image. Determining the pulse condition based on the color Doppler image and the blood flow waveform image.

### CROSS-REFERENCE TO RELATED APPLICATION

This application claims the priority benefit of Taiwan application serial no. 107135144, filed on Oct. 4, 2018. The entirety of the above-mentioned patent application is hereby incorporated by reference herein and made a part of this specification.

### BACKGROUND OF THE INVENTION

### Field of the Invention

The invention relates to a pulse-taking technique, and particularly relates to an ultrasonic-based pulse-taking device and a pulse-taking method.

### Description of Related Art

When a Traditional Chinese Medicine (TCM) physician interviews a patient, the TCM physician obtains information of the patient through four ways of diagnosis including “observation, auscultation and olfaction, inquiry and pulse-feeling”, where “pulse-feeling” is pulse-taking. When pulse-taking is performed, the TCM physician generally feels a pulse condition (i.e. a state of the pulse) of the patient by palpation. However, since the pulse condition obtained through the palpation cannot be converted into quantifiable information, the TCM physician can only judge the pulse condition by his own experience. Such pulse diagnosis method is often easy to cause people's doubt.

In recent years, many researchers have designed various types of pulse-taking devices, though these pulse-taking devices cannot measure enough information to determine all types of pulse conditions. Therefore, these pulse-taking devices are still in the stage of clinical trials and cannot be widely used.

### SUMMARY OF THE INVENTION

In order to obtain enough information to accurately determine various types of pulse conditions, the invention is directed to an ultrasonic-based pulse-taking device and a pulse-taking method.

The invention provides an ultrasonic-based pulse-taking device, which is adapted to determine a pulse condition of a person. The pulse-taking device includes a storage unit, an ultrasonic sensor and a processing unit. The storage unit stores a plurality of modules. The ultrasonic sensor senses a blood vessel of the person to generate a color Doppler image and a blood flow waveform image. The processing unit is coupled to the storage unit and the ultrasonic sensor, and accesses and executes the modules stored in the storage unit. The modules stored in the storage unit include a computation module. The computation module determines the pulse condition according to the color Doppler image and the blood flow waveform image.

The invention provides an ultrasonic-based pulse-taking method, which is adapted to determine a pulse condition of a person. The pulse-taking method includes: sensing a blood vessel of the person to generate a color Doppler image and a blood flow waveform image; and determining the pulse condition according to the color Doppler image and the blood flow waveform image.

Based on the above description, the invention adopts the ultrasonic technique to determine related information of the pulse condition, so as to provide scientific pulse-taking information.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of an ultrasonic-based pulse-taking device according to an embodiment of the invention.

FIG. 2 is a flowchart illustrating an ultrasonic-based pulse-taking method according to an embodiment of the invention.

FIG. 3 is a schematic diagram of a blood flow waveform image according to an embodiment of the invention.

FIGS. 4A and 4B are schematic diagrams of blood flow waveform images according to another embodiment of the invention.

FIG. 5 is a schematic diagram of a color Doppler image according to an embodiment of the invention.

FIG. 6A is a flowchart illustrating a method for determining vascular elasticity according to an embodiment of the invention.

FIG. 6B is a schematic diagram of a waveform curve of the blood waveform diagram of a radial artery and a second derivative curve corresponding to the waveform curve according to an embodiment of the invention.

### DESCRIPTION OF EMBODIMENTS

In order to provide quantifiable pulse-taking information, the invention provides an ultrasonic-based pulse-taking device and a pulse-taking method. The spirit of the invention is conveyed with reference of following content.

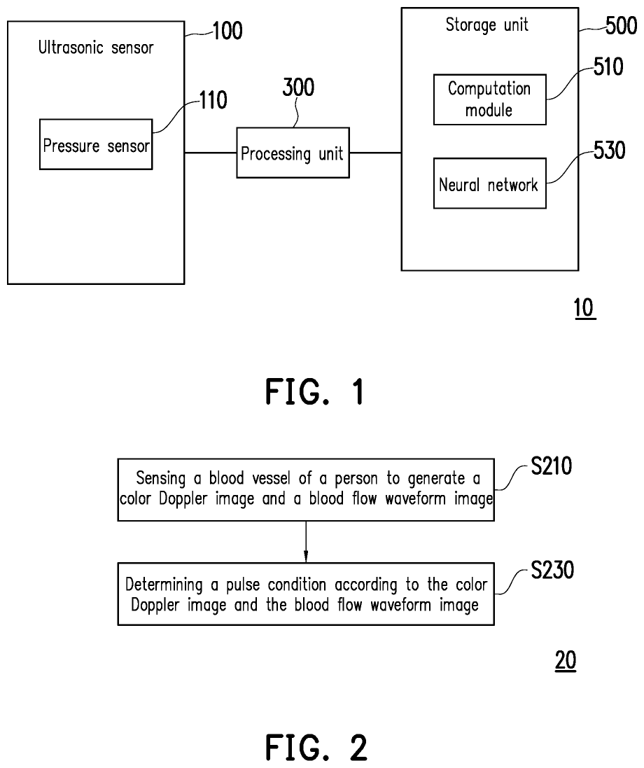

FIG. 1 is a schematic diagram of an ultrasonic-based pulse-taking device 10 according to an embodiment of the invention. The pulse-taking device 10 is adapted to determine a pulse condition of a person. Generally, the pulse condition is determined according to a pulse of a radial artery of the person, though the pulse-taking device 10 of the invention may also be adapted to the pulse condition of any type of blood vessels. The pulse-taking device 10 may include an ultrasonic sensor 100, a processor 300 and a storage unit 500.

The ultrasonic sensor 100 is, for example, a medical ultrasonic sensor, or any sensor adapted to generate a color Doppler image and a blood flow waveform image through the ultrasonic technique. In order to control a pressure applied to a tested part of human body when the ultrasonic sensor 100 contacts the tested part of human body, in some embodiments, the ultrasonic sensor 100 may further include a pressure sensor 110 used for measuring the pressure applied to the tested part by the ultrasonic sensor 100.

The processing unit 300 is coupled to the ultrasonic sensor 100 and the storage unit 500, and is adapted to access and execute a plurality of modules stored in the storage unit 500. The processing unit 300 is, for example, a Central Processing Unit (CPU), or other programmable general purpose or special purpose microprocessor, a Digital Signal Processor (DSP), a programmable controller, an Application Specific Integrated Circuit (ASIC) or other similar device or a combination of the above devices.

The storage unit 500 is used for storing various software, data and various program codes required for operation of the pulse-taking device 10. The storage unit 500 is, for example, any type of a fixed or movable Random Access Memory (RAM), a Read-only Memory (ROM), a flash memory, a Hard Disk Drive (HDD), a Solid State Drive (SSD) or a similar device or a combination of the above devices.

In the embodiment, the storage unit 500 may store a computation module 510. In some embodiments, the storage unit 500 may further store a neural network 530. Functions of the computation module 510 and the neural network 530 are described later.

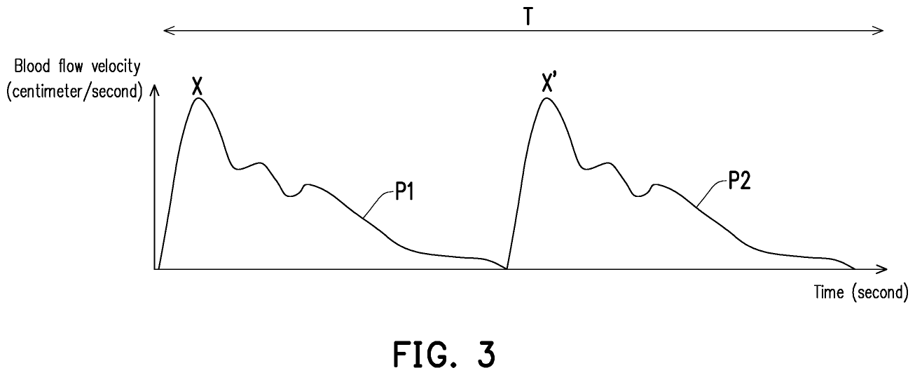

FIG. 2 is a flowchart illustrating an ultrasonic-based pulse-taking method 20 according to an embodiment of the invention. The pulse-taking method 20 is adapted to determine a pulse condition of a person, and is adapted to be implemented by the pulse-taking device 10 of FIG. 1.

In step S210, the pulse-taking device 10 senses a blood vessel of the person to generate a color Doppler image and a blood flow waveform image.

In step S230, the computation module 510 determines a pulse condition according to the color Doppler image and the blood flow waveform image. The pulse condition may include a vascular position and depth, a pulse rate, a pulse strength, a pulse rhythm or a vascular elasticity, though the invention is not limited thereto. To be specific, the blood flow waveform image may be used for determining the pulse conditions such as the vascular position and depth, the pulse rate, the pulse strength or the pulse rhythm, etc., and the color Doppler image may be used for determining pulse conditions such as the vascular position and depth or the vascular elasticity, etc., though the invention is not limited thereto.

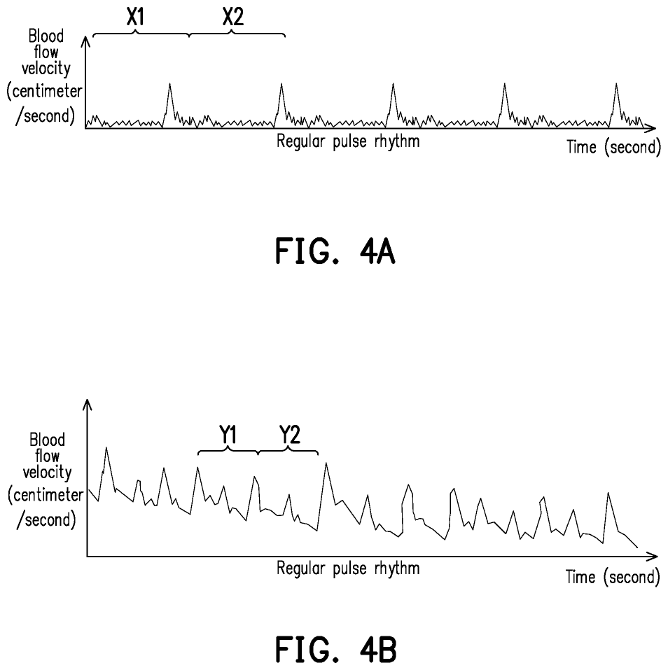

FIG. 3 is a schematic diagram of the blood flow waveform image according to an embodiment of the invention. The blood flow waveform image of FIG. 3 illustrates waveform curves of two pulses, which are respectively a waveform curve P1 and a waveform curve P2 representing a blood flow velocity. The computation module 510 may determine the pulse rate according to the number of the waveform curves generated within a measuring time (the number of the waveform curves/the measuring time). For example, if two waveform curves (i.e. the waveform curve P1 and the waveform curve P2) are generated within a measuring time T, the computation module 510 may determine the pulse rate as 2/T (unit: times/second), which may be converted into a commonly used unit of the pulse rate as 2/T*60 (unit: times/minute). The number of the waveform curves may be obtained based on the number of times that peak values (for example, X and X′ of FIG. 3) appear, which is not limited by the invention. In some embodiments, the pulse-taking device 10 may train the neural network 530 according to historical data, such that the computation module 510 may determine the pulse rate according to the blood flow waveform image and the neural network 530.

Moreover, the blood flow waveform image of FIG. 3 may also be used for determining the pulse strength. The computation module 510 may determine the pulse strength according to the peak values of the waveform curves in the blood flow waveform image. For example, the computation module 510 may obtain the peak value X (unit: centimeter/second) of the waveform curve P1 through the ultrasonic sensor 100, and convert the peak value X (unit: centimeter/second) into the corresponding pulse strength. In some embodiments, the pulse-taking device 10 may train the neural network 530 according to historical data, such that the computation module 510 may determine the pulse strength according to the blood flow waveform image and the neural network 530.

Moreover, the blood flow waveform image of FIG. 3 may also be used for determining a vascular position (for example, Chon, Gwan and Check in the TCM domain) and depth (floating, moderate, sinking in the TCM domain). Generally, the pulse strengths measure at Chon, Gwan and Ckeck are stronger. Therefore, the computation module 510 may determine whether the vascular position sensed by the ultrasonic sensor 100 is at Chon, Gwan and Ckeck according to the peak values of the waveform curves in the blood flow waveform image. On the other hand, the vascular depth may also influence the peak values of the waveform curves. Generally, the deeper the vascular position is, the weaker the measured pulse strength is. Conversely, the shallower the vascular position is, the stronger the measured pulse strength is. Therefore, the computation module 510 may determine whether the vascular depth sensed by the ultrasonic sensor 100 is in a floating, moderate or sinking state according to the peak values of the waveform curves in the blood flow waveform image. In some embodiments, pulse-taking device 10 may train the neural network 530 according to historical data, such that the computation module 510 may determine the pulse rhythm and the vascular position and depth according to the blood flow waveform image and the neural network 530.

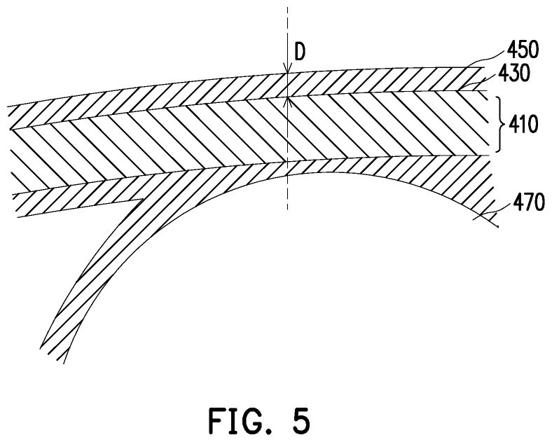

FIGS. 4A and 4B are schematic diagrams of blood flow waveform images according to another embodiment of the invention. FIGS. 4A and 4B respectively illustrate blood flow waveform images when the pulse rhythm is regular and when the pulse rhythm is irregular. The computation module 510 may determine the pulse rhythm according to a waveform of the pulse. For example, a waveform curve X1 and a waveform curve X2 respectively representing two pulses in FIG. 4A have similar waveforms (for example, periods and blood flow velocities of the waveform curve X1 and the waveform curve X2 are similar). Therefore, the computation module 510 may determine that the pulse rhythm is regular based on the fact that the measured pulse waveform curves are similar. On the other hand, a waveform curve Y1 and a waveform curve Y2 respectively representing two pulses in FIG. 4B have different waveforms (for example, periods and blood flow velocities of the waveform curve Y1 and the waveform curve Y2 are quite different). Therefore, the computation module 510 may determine that the pulse rhythm is irregular based on the fact that the measured pulse waveform curves are different. In some embodiments, the pulse-taking device 10 may train the neural network 530 according to historical data, such that the computation module 510 may determine the pulse rhythm according to the blood flow waveform image and the neural network 530.

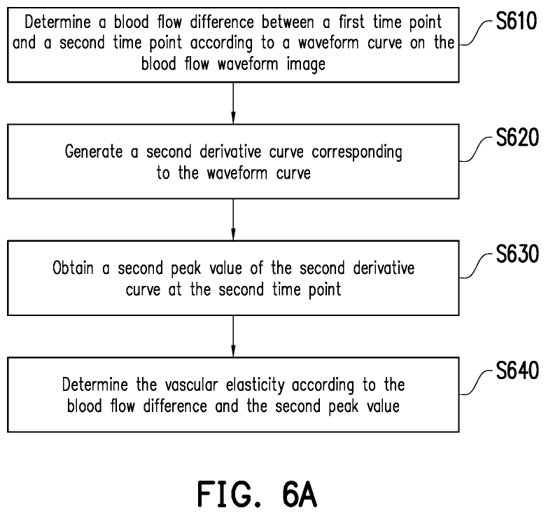

FIG. 5 is a schematic diagram of a color Doppler image according to an embodiment of the invention. The color Doppler image of FIG. 5 illustrates different parts including a radial artery 410, a vascular wall 430, skin epidermis 450 and a radius 470, etc. Based on different blood flow directions, the radial artery 410 in the color Doppler image may present a red color or a blue color. The computation module 510 may determine the vascular position and depth according to a distance between the skin epidermis and the vessel. For example, the computation module 510 may determine the vascular position and depth according to a distance D between the skin epidermis 450 and the vascular wall 430. In some embodiments, the pulse-taking device 10 may train the neural network 530 according to historical data, such that the computation module 510 may determine the vascular position and depth according to the color Doppler image and the neural network 530.

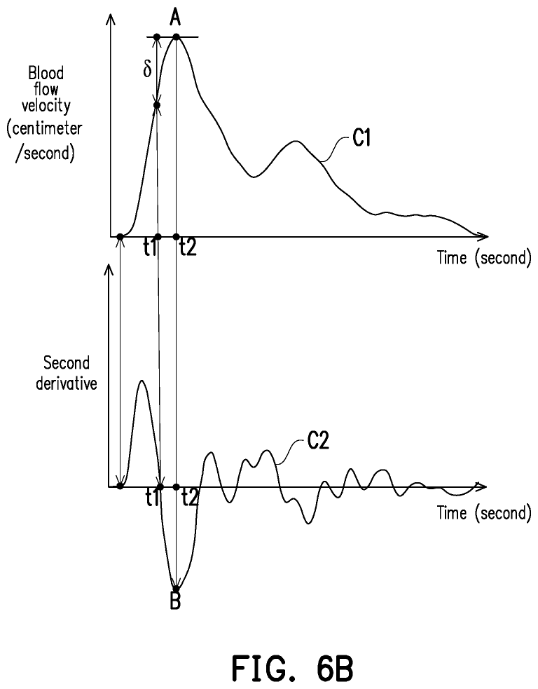

FIG. 6A is a flowchart illustrating a method for determining vascular elasticity according to an embodiment of the invention. FIG. 6B is a schematic diagram of a waveform curve C1 of the blood waveform diagram of the radial artery and a second derivative curve C2 corresponding to the waveform curve C1 according to an embodiment of the invention. FIG. 6A and FIG. 6B may assist understanding the flow of determining the vascular elasticity.

In step S610, the computation module 510 determines a blood flow difference δ between a first time point t1 and a second time point t2 according to the waveform curve C1 on the blood flow waveform image, where the second time point t2 is a time point that the waveform curve C1 reaches a first peak value A. In step S620, the computation module 510 generates the second derivative curve C2 corresponding to the waveform curve C1. In step S630, the computation module 510 obtains a second peak value B of the second derivative curve C2 at the second time point t2. In step S640, the computation module 510 determines the vascular elasticity according to the blood flow difference δ and the second peak value B. To be specific, the computation module 510 may determine a coefficient K of the vascular elasticity during a period between the first time point T1 and the second time point t2 according to a following equation (1), where B is the peak value of the second derivative curve C2 of the waveform curve C1 on the blood flow waveform image at the second time point t2, and δ is the blood flow difference on the waveform curve C1 of the blood flow waveform image between the first time point t2 and the second time point t2.

K=B/δ equation (1)

A following table 1 records meanings of the coefficient K of the vascular elasticity calculated according to the equation (1).

TABLE 1

| Coefficient K of | Abnormality degree of |
|---|---|
| vascular elasticity | vascular elasticity |
| >1.978 | Just as well |
| 1.672~1.978 | Normal |
| 1.511~1.672 | Slight abnormal |
| 1.047~1.511 | Moderate abnormal |
| <1.047 | Severe abnormal |

In summary, the invention adopts the ultrasonic technique to determine related information of the pulse condition. Based on the color Doppler image and the blood flow waveform image generated by the ultrasonic sensor, many pulse conditions including the vascular position and depth, the pulse rate, the pulse strength, the pulse rhythm and the vascular elasticity of the user may be accurately determined. In this way, information of the pulse conditions may be scientifically quantified, so as to improve people's trust in pulse diagnosis.

It will be apparent to those skilled in the art that various modifications and variations can be made to the structure of the invention without departing from the scope or spirit of the invention. In view of the foregoing, it is intended that the invention cover modifications and variations of this invention provided they fall within the scope of the following claims and their equivalents.

What is claimed is:

1. An ultrasonic-based pulse-taking device, adapted to determine a pulse condition of a person, and comprising:
a storage unit, storing a plurality of modules;
an ultrasonic sensor, sensing a blood vessel of the person to generate a color Doppler image and a blood flow waveform image; and
a processing unit, coupled to the storage unit and the ultrasonic sensor, and accessing and executing the modules stored in the storage unit, wherein the modules comprise:
a computation module, determining the pulse condition according to the color Doppler image and the blood flow waveform image.

2. The ultrasonic-based pulse-taking device as claimed in claim 1, wherein the pulse condition comprises at least one of a vascular position and depth, a pulse rate, a pulse strength, a pulse rhythm and a vascular elasticity.

3. The ultrasonic-based pulse-taking device as claimed in claim 2, wherein the step of determining the pulse condition according to the color Doppler image and the blood flow waveform image comprises:
determining at least one of the vascular position and depth and the vascular elasticity according to the color Doppler image.

4. The ultrasonic-based pulse-taking device as claimed in claim 2, wherein the step of determining the pulse condition according to the color Doppler image and the blood flow waveform image comprises:
determining at least one of the vascular position and depth, the pulse rate, the pulse strength, and the pulse rhythm according to the blood flow waveform image.

5. The ultrasonic-based pulse-taking device as claimed in claim 2, wherein the computation module determines the vascular elasticity according to following steps:
determining a blood flow difference between a first time point and a second time point according to a waveform curve on the blood flow waveform image, wherein the second time points is a time point when the waveform curve reaches a first peak value;
generating a second derivative curve corresponding to the waveform curve;
obtaining a second peak value of the second derivative curve at the second time point; and
determining the vascular elasticity according to the blood flow difference and the second peak value.

6. The ultrasonic-based pulse-taking device as claimed in claim 2, wherein the computation module determines the vascular position and depth according to a distance between skin epidermis and the blood vessel.

7. The ultrasonic-based pulse-taking device as claimed in claim 2, wherein the modules further comprise:
a neural network, wherein the computation module determines the pulse condition according to the neural network, the blood flow waveform image, and the color Doppler image.

8. An ultrasonic-based pulse-taking method, adapted to determine a pulse condition of a person, and comprising:
sensing a blood vessel of the person to generate a color Doppler image and a blood flow waveform image; and
determining the pulse condition according to the color Doppler image and the blood flow waveform image.

9. The ultrasonic-based pulse-taking method as claimed in claim 8, wherein the pulse condition comprises at least one of a vascular position and depth, a pulse rate, a pulse strength, a pulse rhythm and a vascular elasticity.

10. The ultrasonic-based pulse-taking method as claimed in claim 9, wherein the step of determining the pulse condition according to the color Doppler image and the blood flow waveform image comprises:
determining at least one of the vascular position and depth and the vascular elasticity according to the color Doppler image.

11. The ultrasonic-based pulse-taking method as claimed in claim 9, wherein the step of determining the pulse condition according to the color Doppler image and the blood flow waveform image comprises:
determining at least one of the vascular position and depth, the pulse rate, the pulse strength and the pulse rhythm according to the blood flow waveform image.

12. The ultrasonic-based pulse-taking method as claimed in claim 9, wherein the step of determining the pulse condition according to the color Doppler image and the blood flow waveform image comprises:
determining a blood flow difference between a first time point and a second time point according to a waveform curve on the blood flow waveform image, wherein the second time points is a time point when the waveform curve reaches a first peak value;
generating a second derivative curve corresponding to the waveform curve;
obtaining a second peak value of the second derivative curve at the second time point; and
determining the vascular elasticity according to the blood flow difference and the second peak value.

13. The ultrasonic-based pulse-taking method as claimed in claim 9, wherein the step of determining the pulse condition according to the color Doppler image and the blood flow waveform image comprises:
determining the vascular position and depth according to a distance between skin epidermis and the blood vessel.

14. The ultrasonic-based pulse-taking method as claimed in claim 9, wherein the step of determining the pulse condition according to the color Doppler image and the blood flow waveform image comprises:
determining the pulse condition according to a neural network, the blood flow waveform image and the color Doppler image.
